(19) United States
(12) Patent Application Publication
(10) Pub. No.: US 2009/0012469 A1
(43) Pub. Date: Jan. 8, 2009

(54) Methods and devices for obesity treatment

(76) Inventor: Henry Nita, Redwood Shores, CA (US)

Correspondence Address: Raymond Sun, 12420 Woodhall Way, Tustin, CA, 92782, US

(21) Appl. No.: 12/008,343

(22) Filed: Jan. 10, 2008

Related U.S. Application Data
(60) Provisional application No. 60/958,648, filed on Jul. 6, 2007.

(51) Int. Cl. A61M 29/00; A61F 7/12; A61M 25/04

(52) U.S. Cl. 604/104; 604/113; 604/174

(57) ABSTRACT

A method for inducing malabsorption in a small intestine of a patient may involve advancing a portion of a catheter into the patient's small intestine and expanding a portion of the catheter to apply force to the small intestine's inner wall to modify mucosal villi and/or crypts. The method for causing weight loss in a patient may also involve dilating at least a portion of the patient's small intestine to modify mucosal villi and/or crypts, destroying mucosal villi and/or crypts of the patient's small intestine using a combined coaxial and radial catheterization of the intestine, or isolating a length of the patient's small intestine and delivering a substance to the isolated length to destroy mucosal villi and/or crypts.

### CROSS REFERENCE TO RELATED APPLICATIONS

The present application claims priority to U.S. Provisional Patent Application Ser. No. 60/958,648, entitled “Methods and Devices for Obesity,” and filed on Jul. 6, 2007, the full disclosure of which is hereby incorporated by reference.

### FIELD OF THE INVENTION

The present invention relates to medical/surgical methods and devices. More specifically, the present invention relates to methods and devices for treating obesity.

### BACKGROUND OF THE INVENTION

Obesity is a major health problem, especially in the United States but also in other developed and developing countries. Morbid obesity is an extreme form of obesity, generally defined as a body mass index (BMI)≧40 kg/m² and often accompanied with one or more associated diseases such as diabetes and heart disease. While the basic mechanism of obesity is an imbalance between caloric intake and burn rate, underlying causative factors are varied and complex. Therefore, conservative attempts at sustained weight loss in the morbidly obese population are almost always unsuccessful. Often, there are genetic and other biological influences that may override environmental causes. Consequently, obesity is a disease that eludes simple treatment, with a recurrence rate above 90% for those who attempt to lose weight. Moreover, long-term results using conservative treatments for morbid obesity are generally unsuccessful and are typically associated with further loss of self-esteem as weight is regained. Hypertension, cardiovascular disease, diabetes, along with a host of other co-morbidities, all makes morbid obesity second only to smoking as a preventable cause of death.

Surgical procedures to treat obesity date back to 1889 (Billroth), with the earliest peer reviewed procedure being the jejuno-ileal bypass in 1954 (Kreman). A successful procedure is commonly defined as one that results in at least 50% excess weight loss at two years.

Currently available obesity surgery procedures can generally be divided into two categories. One category is restrictive procedures, in which the intervention provides the patient with a sense of satiety upon ingestion of a smaller volume of food than without the procedure. A second category is malabsorptive procedures, whereby food absorption and resultant caloric intake is partially inhibited, typically by modulating the small or large intestine physiology. Currently, the most commonly performed surgical obesity procedures is the Roux-en-Y gastric bypass (RYGB). This procedure includes both restrictive and Malabsorptive elements. Other forms of bariatric surgery include the Fobi pouch, bilio-pancreatic diversion, and gastroplasty or “stomach stapling”. The single existing procedure that involves an implanted device is the Lap-Band—a laparoscopically installed inflatable cuff placed around the top of the stomach just below the lower esophageal sphincter (LES). This Lap-band device affects satiety only and, therefore, is unlikely to be as effective as other surgical procedures that also effect absorption.

The RYGB procedure facilitates the movement of the jejunum to a high position by using a retrocolic Roux-en-Y loop. The procedure is generally performed through a 6-8 inch incision extending from the end of the breastbone to just above the navel. The stomach is completely divided into two unequal (upper and lower) pouches, using an automatic stapling device with the raw surface reinforced with additional sutures. The upper pouch typically measures less than about 1 ounce or 20 cc, while the lower larger pouch remains generally intact and continues to secrete stomach juices flowing through the intestinal tract. A segment of the small intestine (just distal of the duodenum or proximal of the jejunum) is then brought from the lower abdomen and joined with the upper pouch to form an end-to-end anastomosis created through a half-inch opening, also called the stoma. This segment of the small intestine is called the “Roux loop” and carries food from the upper pouch to the remainder of the intestines, where the food is digested. The remaining lower pouch and the attached segment of duodenum are then reconnected to form another anastomotic connection to the Roux loop at a location approximately 50-150 cm from the stoma, typically using a stapling instrument. It is at this connection that the digestive juices from the bypassed stomach, pancreas, and liver enter the jejunum or ileum to aid in the digesting of food. Therefore, in this procedure, 50 cm to 150 cm of the proximal small intestine is bypassed from the digestive track, thereby causing malabsorption and reducing caloric absorption. Also, due to the small size of the upper pouch, patients are forced to eat at a slower rate and are satiated much more quickly, thereby reducing the caloric intake.

Although they can sometimes be an effective obesity treatment, the RYGB procedure and other surgical obesity procedures are highly invasive and potentially dangerous to the patient. The procedures are typically very time consuming and require general anesthesia, and post-operative recovery is typically a very long, painful process. Complications during such surgical procedures and side effects after them, such as uncontrollable diarrhea, electrolyte imbalance, unpredictable weight loss, and reflex of nutritious chime proximal to the site of anastomosis, can have a significant negative impact on the quality of life. In the worst cases, death may result. At the same time, however, patients are increasingly requesting such procedures when they cannot lose weight by other methods, and obesity remains an enormous medical problem without a known cure. Therefore, there is a need for less invasive, lower risk interventions to treat obesity.

### BACKGROUND REFERENCES

The following references are cited as background only: U.S. Patent Application Pub. Nos. 2006/0240096 and 2006/0086362; U.S. Pat. Nos. 4,315,509, 4,694,827, 4,723,547, 6,063,056, 6,200,256, 6,258,099 and 7,066,905; and European Patent No. EP1462116.

### SUMMARY OF THE INVENTION

Various embodiments of the present invention provide devices and methods to alter the effective absorption area of the gastrointestinal tract, including the mucosa of the small intestine. Various embodiments may be used to reduce the surface area of intestinal mucosa by removing, damaging or destroying villi and/or crypts, which may cause malabsorption and, thus, weight loss.

In some embodiments, a device may be introduced into the small intestines of the patient through the mouth or nostril, pharynx, esophagus or stomach with fluoroscopic guidance or by using an endoscope. Such embodiments may involve delivery by any of a variety of different approaches, including, but not limited to, transesophageal, endoscopic, laparoscopic and/or surgical approaches. For example, in various alternative embodiments, delivery may be accomplished by an endoscopically delivered catheter, a laparoscopically inserted intraintestinal catheter, and/or a surgically inserted intraintestinal catheter. The devices and methods described herein may be used to modify villi structure by delivering a catheter coaxially or transluminaly through the small intestines. Once the catheter is located at the targeted intestinal site, the device may apply tangential, normal (orthogonal, perpendicular) or combination forces to the wall of the small intestines. Such forces may cause the villi and/or crypts to be compressed, compacted, cut, squeezed, damaged and/or removed. The forces on the intestinal wall may be applied in a radial or longitudinal direction with respect to the intestine. For example, an inflated balloon or cuff (either a circumferential or non-circumferential balloon) can damage the mucosal villi, resulting in their obliteration by the compression forces exerted on the intestinal wall during inflation or expansion.

In various embodiments, the method of destroying or reducing the function of villi by damaging its surface using such devices and methods may start at any location within the small intestines and proceed toward the duodenum or toward the jejunum. Typically, the length of damage to the intestinal wall will be limited to a desired length, such as but not limited to between about 50 cm and about 150 cm. In various embodiments, the starting point of tissue damage may be located, for example, from about 10 cm to about 300 cm or more distal to the duodenum. In various embodiments, the treated intestinal area may be continuous or multiple discrete areas. Because mucosal villi and/or crypts may regenerate over time, in some embodiments the method may be repeated to produce desired results. To reduce the re-growth rate of the villi, it may be desirable to also damage the crypts during treatment.

In one embodiment, a catheter having a distal end, a proximal end and at least one lumen extending longitudinally through the catheter may be introduced into a patient's small intestine. In some embodiments, the catheter may include a polymeric tubular structure with metallic reinforcement such as a braid, a coil or combination of both for preventing kinking. A y-connector may be located on the proximal end of the catheter. The distal end or tip of the catheter may have an enlarged bulbous shape (i.e. balloon or cuff) made of polymer, rubber, metal or combinations thereof, configured to damage, cut or destroy villi. In some embodiments, the distal tip of the catheter may be expandable to allow insertion of the catheter into the small intestine with a lower distal tip profile and expansion of the distal tip during the procedure. Optionally, the catheter's distal tip may include cutting edges, protrusions, various surface textures and/or other ablative features. In some embodiments, catheter length should be sufficient to extend through the oral cavity, esophagus, gastric cavity, into the small intestine and at least 300 cm distal to the duodenum. The catheter may be placed inside the small intestine using either a guidewire, a sheath, an endoscope's working channel, or a separate lumen of an endoscope.

Some embodiments may include one or more additional devices to serve as an anchor to hold intestines or to limit intestinal movement during positioning of the therapy catheter. One example of such an anchoring device may be a balloon catheter that may be deployed either distally or proximally to the therapeutic device. A therapy catheter may be introduced through the anchoring balloon catheter and positioned at a desired treatment location within the intestine.

In some embodiments, villi destruction and altering crypts may be accomplished by frictional forces between the device and the small intestine generated while moving the deployed catheter's tip with respect to the mucosal villi, thereby causing villi and/or crypts eradication. The catheter movement within the small intestines may be repeated until satisfactory villi/crypts destruction is achieved.

In an alternative embodiment, the catheter may be configured to deliver a pressurized liquid and/or to transmit energy such as radiation, laser or ultrasound to tissue. The catheter tip may alternatively be heated, using low electrical current, microwave or radio-frequency energy to further enhance villi/crypts destruction. In various embodiments, the tip of the catheter may have any of a variety of expandable shapes and configurations, including but not limited to an expandable metallic construction including a braid or mesh or a circumferential or non-circumferential balloon shape. In another embodiment, the expandable element may have a cork-screw shape. Such a device, once expanded, may be rotated at the catheter's proximal end to advance the cork-screw device through a targeted length of intestine. The outer edges of the expandable element could have harder, rougher and/or sharper edges to allow for controlled damage of the intestinal villi. Alternatively, as the cork-screw device is advanced, it may drag behind it a circumferential element that damages or removes some or all of the villi.

To prevent unwanted damage to the intestine, such as perforation, in some embodiments, the device may be limited in the amount of pressure it may exert against an intestinal wall. For example, in some balloon expandable embodiments, forces may be limited to about 30 psi normal forces to the intestinal wall with a preferred load of less than about 10 psi. In some embodiments, the amount of applied pressure may be monitored and controlled to control the amount or depth of damage to the intestine. In one embodiment, perhaps to make the treatment more permanent, the amount of pressure may be controlled to induce enough damage to the mucosa to cause scar formation rather than new villi formation. Alternatively, since the intestine is a virtual space and is collapsed when in its relaxed state, to add additional control to the procedure it may be desirable to first measure the diameter of the intestine at a given pressure using a compliant or oversized device (or alternative means). Following the diameter measurement an expandable balloon (complaint, semi-compliant or non-complaint balloon) can be dilated to a larger diameter than initially measured. Generally, however, in embodiments in which the device includes an expandable cuff, the maximum expanded size should not significantly exceed the small intestine's internal diameter when inflated to a pressure near its rupture point.

In another embodiment, a balloon catheter may be used to focally dilate and destroy/damage villi and/or crypts. In various embodiments, the balloon may be made of a generally elastic material such as elastomer, silicone, latex or other compliant or non-compliant polymer. The balloon may have any suitable dimensions, such as a deflated diameter that allows the device to fit within a delivery tube and an inflated diameter of between about 0.5 cm and about 8 cm, and more preferably between about 2.0 cm and about 5.0 cm. In various embodiments, the balloon may have a cylindrical, spherical or other shape. Optionally, the distal end of the balloon may have a conical profile to allow easy insertion.

In one embodiment, the balloon catheter may include a shaft which has one or more lumens and can be used as an over-the-wire, fixed wire, rapid exchange, semi-movable wire catheter, changeable from one to another at any time, or have no guidewire delivery option at all. A fluid (liquid or gas) may be used to inflate and deflate the balloon. In some embodiments, the balloon length may be about 2-50 cm and preferably about 10-20 cm. The balloon structure may include any of a variety of external surface configurations, hard or sharp edges, and/or textures to facilitate villi destruction during dilation. Such shapes may include protrusions, texture, reinforcements and/or cutting elements. In some embodiments, the surface of the balloon may be embedded with metallic or stiffer polymer materials to further facilitate villi and/or crypts destruction as well. In various embodiments, the dilatation process may take from a few seconds to minutes or even hours, and may be repeated as many times as needed to achieve desired results. Dilatation may induce compression, cutting, smashing, crushing, crashing, tearing, squeezing, flattening and/or shearing of mucosal villi.

In some embodiments, an endoscope may be used to verify villi destruction. The balloon may be reinforced with a metallic material to improve its endurance. Such a reinforcement feature, in some embodiments, may also be configured to heat the surface of the balloon, such as to a temperature above about 45-50° C., to potentially cause additional degeneration and cellular damage of mucosal villi and crypts. In one embodiment, the balloon may have infusion pores to deliver drugs to the intestinal wall. Various agents that might be delivered include but are not limited to alcohol, chemotherapy agents, paxlitaxol, corticosterone, Mitomycin C, Phenol and other agents that may inhibit cell proliferation in the intestine. In some embodiments, the therapeutic catheter may be rapidly oscillated back and forth using ultrasound energy at the proximal or distal end of the catheter to assist in damaging and/or removing villi. The balloon may also be made of non-compliant or semi-compliant polymers. In such a case, the size of the intestinal channel may be assessed prior to the procedure to assure appropriate balloon size selection.

In another embodiment, a combination approach utilizing longitudinal and/or radial forces about the internal wall of the intestine may be used to eradicate mucosal villi and/or crypts. For example, the catheter with a balloon or other expandable tip may be positioned against the intestinal wall first to induce an initial damage to villi in the area of treatment. Then, the catheter may be repositioned by moving back and forth and/or by rotating the distal end of the catheter anywhere between about 10-360° to foster more mucosal villi destruction. In some instances, more than one 360° rotation may be needed to achieve desired results.

In another embodiment, a device comprising a catheter body and two balloons located on the distal end of the catheter body may be used to help destroy mucosal villi. The catheter body may include a longitudinal shaft with separate lumens to inflate and deflate each balloon and at least one lumen extended longitudinally to deliver one or more therapeutic agents. Balloons may be located on the catheter shaft distal end about 1-50 cm apart from each other, and preferably about 10-20 cm apart from each other. The catheter shaft between balloons may have perforations or holes to deliver therapeutic agents. Such agents may include, but are not limited to hot liquids, cold liquids, cold gases and/or steam to perform a thermal therapy to destroy mucosal villi. Therapeutic agents may also include drugs such as alcohol, paxlitaxol, corticosterone, Mitomycin C, Phenol and/or other agents that inhibit cell proliferation or cause cell aptosis in the intestine. Additional agents may include but are not limited to ethanol, benzalkonium chloride, acetic acid, and/or hydrochloric acid. A neutralizing or diluting agent may be disposed inside the active agent or subsequently delivered to ensure termination of treatment. Neutralizing or diluting agents include but are not limited to an ingestible antacid or water. When both balloons are inflated within the intestine, a space between balloons may be considered a treatment area. Any treatment agent may be delivered into a treatment area and removed after a local therapy to destroy mucosal villi and/or crypts is completed. Some agents that are not causing harm may be left within the intestines. This local therapy of villi destruction may be repeated along the small intestine for as long a distance as desired. Alternatively, with the balloons inflated, microbubbles may be delivered in an aqueous solution and imploded using ultrasound energy from a probe located external to or within the patient's body. When the microbubbles collapse in the vicinity of the intestinal lining, they may cause mechanical trauma, thus injuring or ablating the lining.

In various alternative embodiments, any of the above-mentioned therapies may be performed using infusion catheter(s) without balloon(s), in a single or multi-lumen configuration. Such infusion catheters may be utilized to deliver all mentioned agents to small intestines to destroy villi and/or crypts.

In another alternative embodiment, a device may be used to reduce blood supply to villi microvasculature by selective embolization of a blood supply feeder. In such an embodiment, an infusion catheter having a distal end, a proximal end and at least one lumen extending longitudinally along the catheter may be introduced via femoral, brachial or radial access into the ostium and the superior mesenteric artery. The catheter may then be further advanced into the superior mesenteric artery arcades and then into the ostium of the first few arteries that feed blood to the small intestine between the duodenum and jejunum. Embolization may be achieved using a variety of embolic materials such as glue, alcohol, metal coils and particles.

In various embodiments, the devices and methods as described or in combination with other devices and methods may be used to treat other health conditions associated with the gastrointestinal system or facilitate other gastrointestinal therapies. For example, it may be desirable to use the malabsorptive therapies described herein in combination with a restrictive therapy, pharmacological therapy and/or electrical stimulation therapy to maximize the effectiveness. Examples of restrictive therapies include the Lap-band, balloon implants in the stomach, endoluminal restrictive therapies including restrictive valves near the gastro-intestinal-junction, and remodeling of the stomach. For electrical stimulation therapies, several products are being developed that either provide satiety or modulate gastrointestinal activity through active stimulation of the branch of the vagal nerve or other nerves or organs. Lastly, pharmacological approaches often focus on reducing satiety. The malabsorptive therapies described herein may be used to complement such an approach and facilitate a patient's weight loss.

These and other aspects and embodiments are described more fully below in the Detailed Description, with reference to the attached Drawings.

### BRIEF DESCRIPTION OF THE DRAWINGS

FIG. 1 is a frontal, cross-sectional view of a patient's gastrointestinal tract, showing a device in place to perform a procedure, in accordance with one embodiment of the present invention;

FIG. 2 is a cross-sectional view of a portion of a small intestine and a side view of a distal portion of the balloon catheter device from FIG. 1, with the balloon in its deflated state;

FIG. 3 is a view of the intestine and device of FIG. 2, with the balloon in its inflated state;

FIG. 4 is a view of the intestine and device of FIGS. 2 and 3, demonstrating a method for moving the balloon along the intestine to affect a desired length of the mucosal wall, according to one embodiment of the present invention;

FIG. 5 is a cross-sectional view of a portion of a small intestine and a side view of a distal portion of an expandable distal cuff device in the intestine for performing a procedure, showing flattening of mucosal villi, according to one embodiment of the present invention;

FIG. 6 is a cross-sectional view of a portion of a small intestine and a side view of a distal portion of an expandable distal cuff device in the intestine for performing a procedure, with the device having a distal balloon anchor, according to one embodiment of the present invention; and

FIG. 7 is a cross-sectional view of a portion of a small intestine and a side view of a distal portion of a dual balloon/therapeutic agent delivery device in the intestine for performing a procedure, showing delivery of a therapeutic agent, according to one embodiment of the present invention.

### DETAILED DESCRIPTION OF THE INVENTION

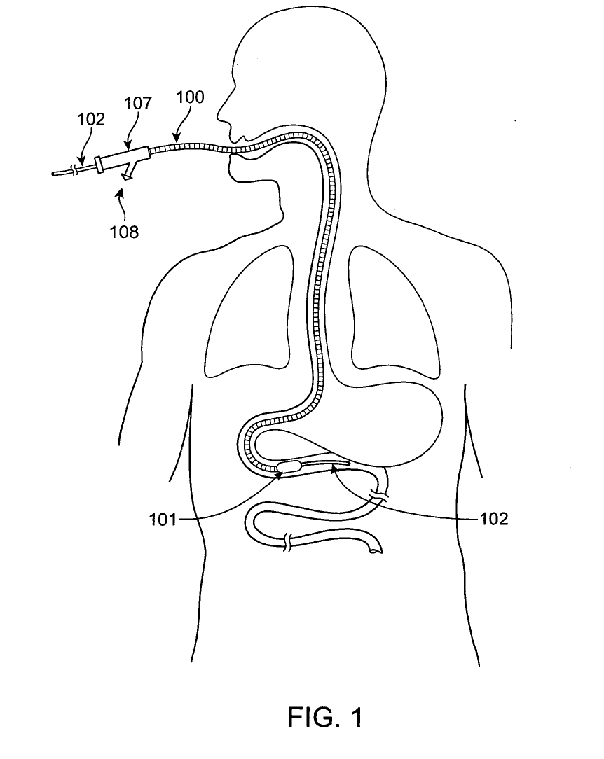

Referring to FIG. 1, one embodiment of a balloon catheter 100 may be delivered through an oral cavity, esophagus and stomach, into the small intestine of a patient. In various embodiments, catheter 100 may be delivered alone, using a guidewire 102 (over the wire, rapid exchange, or the like) and/or through an endoscope (not shown). Catheter 100 may have a length sufficient to extend from outside a patient's mouth to a treatment location within the small intestine. In one embodiment, an expandable balloon 101 may be coupled with catheter 100 at or near its distal end. Balloon 101 may be inflated via any suitable connection to an inflation device, such as a port 108 on a Y-connector 107.

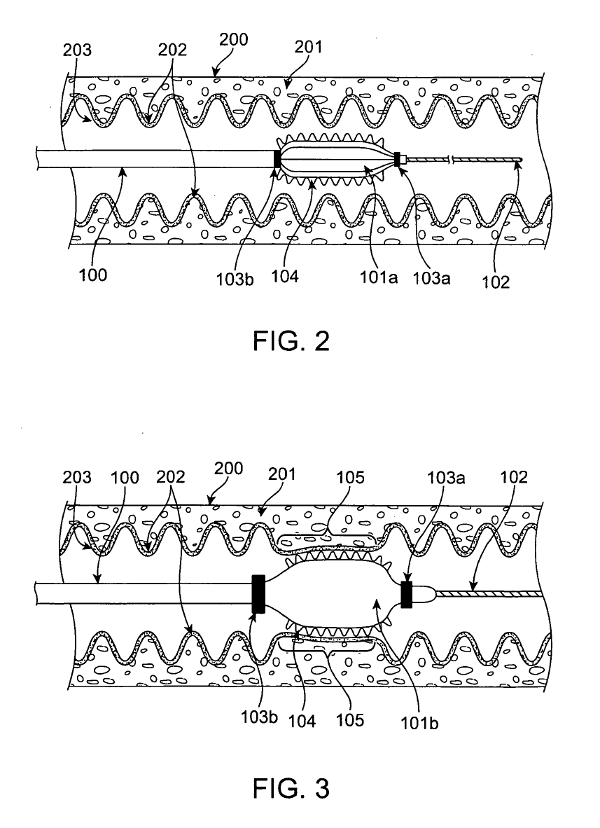

With reference now to FIG. 2, a more detailed view of a small intestine 200, intestinal wall 201 (or “mucosal wall”), mucosal villi 202 and crypts 203, as well as a more detailed view of a distal portion of catheter 100 and balloon 101, are shown. In one embodiment, to facilitate advancement of balloon 101 through the gastrointestinal tract, catheter 100 is introduced over guidewire 102, with balloon 101 in its deflated form 101a. In one embodiment, balloon 101 may include multiple protrusions 104 disposed along its outer surface. Catheter 100 may also include any suitable number of radiopaque markers or materials, such as but not limited to a distal marker 103a and a proximal marker 103b, for facilitating advancement of catheter 100 to a desired location in small intestine 200.

Referring to FIG. 3, catheter 100 is shown with balloon 101 in its inflated form 101b. Inflated balloon 101b is shown smashing (or flattening) mucosal villi 202 and/or crypts 203 against the intestinal wall, inducing destruction of villi 202 and/or crypts 203 within a treatment area 105.

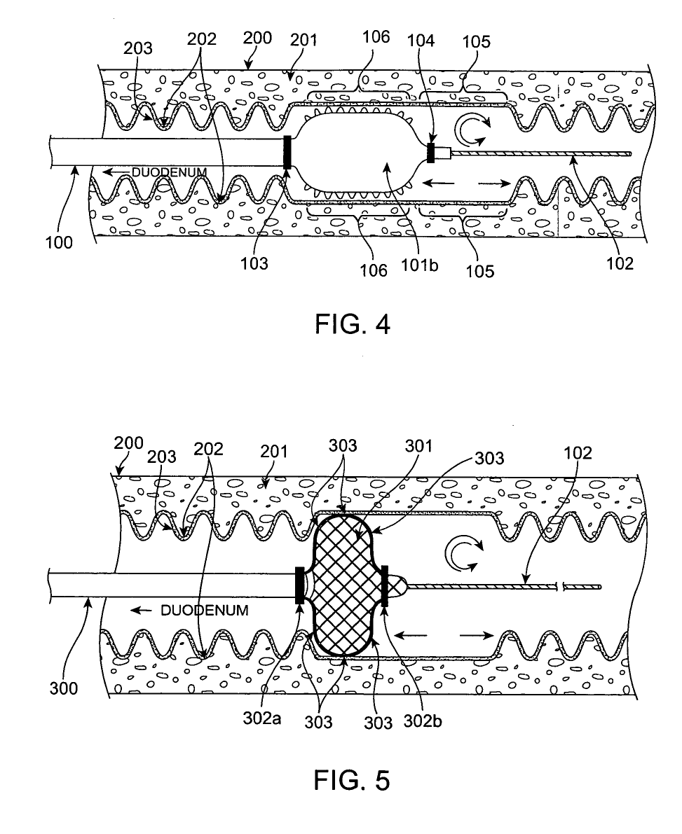

With reference now to FIG. 4, one embodiment of a method for affecting mucosal wall 201 of small intestine 200 may involve moving inflated balloon 101b along a length of small intestine 200. Moving balloon 101b in its inflated state (or alternatively deflating, moving and re-inflating the balloon) allows a treatment to be extended along a length 106, in addition to the length of the treatment area 105 of intestinal wall 201. Such progressive destruction of mucosal villi 202 and/or crypts 203 may continue until a desired length of intestinal wall 201 is dilated. In various embodiments, balloon dilatation may proceed distal from the duodenum or proximal from the jejunum. Balloon 101 may be moved along a length of small intestine 200 in its inflated state 101b, deflated state 101a (as shown in FIG. 2), or a combination of both states.

With reference now to FIG. 5, in an alternative embodiment, a catheter 300 may include an expandable distal cuff 301 (or “tip”), which in one embodiment may be made of metallic or polymeric mesh and may include a distal radiopaque marker 302b and a proximal radiopaque marker 302a. Catheter 300 may be introduced into small intestine 200 with cuff 301 in a non-expanded/lower profile (not shown) alone, over the guidewire 102 and/or using an endoscope (not shown). When distal cuff 301 is advanced to a desired treatment location, distal cuff 301 may be expanded. An outer surface 303 of cuff 301, or a portion thereof, may contact mucosal wall 201 of small intestine 200 during a procedure. In some embodiments, outer surface 303 may include any of a number of surface features, such as protrusions, a heating coil and/or other device(s) to enhance destruction, flattening and the like of villi 202 and/or crypts 203. In some embodiment, expanded distal cuff 301 may be translated back and forth and/or rotated to obliterate mucosal villi 202 and/or crypts 203.

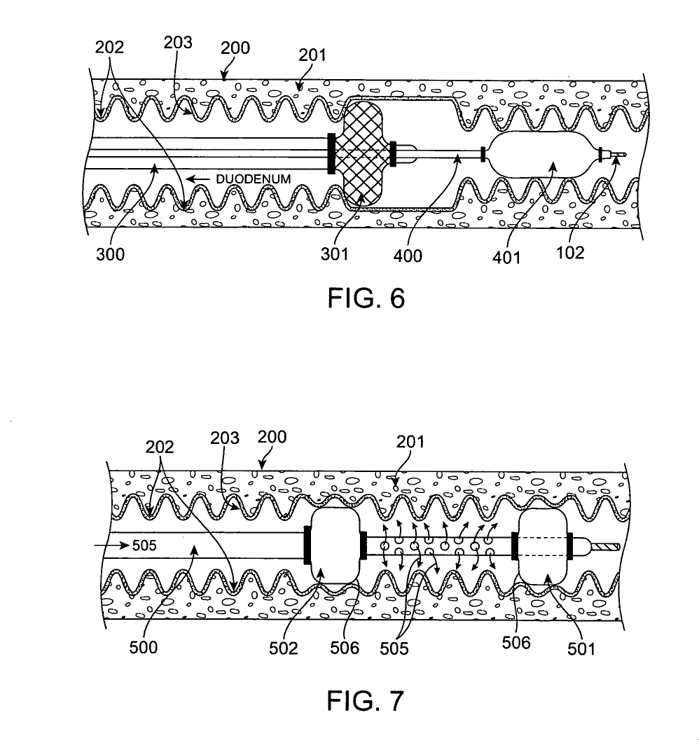

Referring now to FIG. 6, a catheter 300 with expandable cuff 301 is shown, as in FIG. 5, but with the additional, optional feature of a second catheter 400 with a second balloon 401 at or near its distal end, used as an anchor to hold catheter 300 generally in place during a procedure. When second balloon catheter 400 is anchored, it may help hold catheter 300 relatively stable, relative to a portion of small intestine 200. Once anchor balloon 401 is in place and inflated, catheter 300 may be advanced over or through second catheter 400 into small intestine 200. Mucosal villi 202 and/or crypts 203 may then be flattened, destroyed or otherwise affected by inflating balloon 301 and, in some embodiments, by retracting/pulling catheter 300 and balloon 301 proximally, away from second balloon 401 and toward the duodenum. Meanwhile, as catheter 300 and balloon 301 are retracted, second (anchoring) balloon 401 may help hold small intestine 200 relative to catheter 300, to prevent unwanted movement of small intestine 200, relative to the device, during treatment. In an alternative embodiment, when the method involves moving catheter 300 and balloon 301 toward the jejunum, second catheter 400 and second balloon 401 may be configured such that they anchor the device proximally, and catheter 300 and balloon 301 may be pushed (or “advanced”) distally away from anchoring balloon 401 and toward the jejunum.

With reference now to FIG. 7, in another embodiment, a dual balloon catheter 500 may include a distal balloon 501 and proximal balloon 502. A treatment length 506 of the catheter 500, between balloons 501, 502, may include multiple pores 505 (or apertures or holes), through which any suitable substance (or combination of substances) may be delivered to affect the mucosal wall 201. Balloons 501, 502, in their expanded state, typically help to prevent delivered substances from passing proximally or distally beyond desired treatment length 506. In one embodiment, one or more therapeutic agents or other delivered substance(s) may be removed from the intestine after the completion of therapy via application of negative pressure (suction), either through pores 505 or via a separate suction device, lumen or the like.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

What is claimed is:

1. A method for inducing malabsorption in a small intestine of a patient, the method comprising:
advancing a portion of a catheter into the patient's small intestine; and
expanding a portion of the catheter to apply force to the small intestine's inner wall to modify at least one of villi or crypts on a mucosal surface of the small intestine.

2. The method of claim 1, wherein expanding the catheter applies controlled tangential or normal force to limit the damage to the mucosal surface.

3. The method of claim 1, wherein expanding the catheter comprises expanding a tip portion of the catheter.

4. The method of claim 3, wherein the expandable tip is selected from the group consisting of a balloon, a cuff and a mesh.

5. The method of claim 3, wherein the expandable tip comprises a heating element.

6. The method of claim 1, further comprising moving the expanded catheter along a length of the small intestine.

7. The method of claim 1, further comprising rotating the catheter inside the small intestine.

8. The method of claim 2, wherein rotating the catheter causes a portion of the catheter to modify villi by a process selected from the group of compressing, cutting, smashing, crushing, crashing, tearing, squeezing, flattening and shearing.

9. The method of claim 1, further comprising anchoring a portion of the catheter to the inner wall of the small intestine, using one or more anchoring members coupled with the catheter.

10. The method of claim 1, wherein advancing the catheter includes using an endoscope or fluoroscopy to visualize the advancing step.

11. A method for causing weight loss in a patient comprising dilating at least a portion of the patient's small intestine to modify at least one of mucosal villi or crypts in the small intestine.

12. The method of claim 11, wherein dilating the small intestine comprises expanding a balloon catheter.

13. The method of claim 11, wherein dilating the small intestine comprises expanding a cuff.

14. The method of claim 11, wherein dilating the small intestine creates an effect on the small intestine selected from the group consisting of compressing, cutting, smashing, crushing, crashing, tearing, squeezing, flattening and shearing at least one of mucosal villi or crypts.

15. The method of claim 11, further comprising applying heat to a mucosal surface of the small intestine using one or more heating elements coupled with an expandable balloon catheter used to dilate the small intestine.

16. The method of claim 11, further comprising applying heat to a mucosal surface of the small intestine using one or more heating elements coupled with an expandable cuff catheter used to dilate the small intestine.

17. A method for causing weight loss in a patient comprising destroying at least one of mucosal villi or crypts of the patient's small intestine, using a combined coaxial and radial catheterization of the intestine.

18. The method of the claim 17, wherein the coaxial catheterization comprises moving a catheter along the larger axis of the intestinal wall.

19. The method of the claim 17, wherein the radial catheterization comprises dilating at least one of a balloon or cuff.

20. The method of claim 17, wherein the combined coaxial and radial catheterization comprises moving an inflated balloon or cuff catheter along the intestines.

21. The method of claim 17, wherein the coaxial and radial catheterization comprises rotating a catheter against a mucosal wall of the small intestine.

22. A method for causing weight loss in a patient, the method comprising:
isolating a length of the patient's small intestine; and
delivering a substance to the isolated length to destroy at least one of mucosal villi or crypts of the small intestine.

23. The method of claim 22, wherein isolating the length of the small intestine comprises expanding two balloons, coupled with a catheter, within the small intestine to contact an inner wall of the small intestine, wherein the isolated length is located between the duodenum and the jejunum of the small intestine.

24. The method of claim 22, wherein the substance is selected from the group consisting of hot liquid, cold liquid, cold gas, steam and a drug.

25. The method of claim 24, wherein the drug is selected from the group consisting of alcohol, paxlitaxol, corticosterone, Mitomycin C, Phenol and other agents that inhibit cell proliferation in the intestine.
